(19) United States
(12) Patent Application Publication
(10) Pub. No.: US 2022/0047882 A1
(43) Pub. Date: Feb. 17, 2022

(54) Method and the device for diagnostics and therapy based on DNA resonance, a method for locating resonating DNA sequences in the genome, and a method for identifying the properties, structures, mechanisms, and frequencies of electromagnetic resonance in DNA molecules

(71) Applicant: Max Myakishev-Rempel, San Diego, CA (US)

(72) Inventor: Max Myakishev-Rempel, San Diego, CA (US)

(21) Appl. No.: 16/992,080

(22) Filed: Aug. 12, 2020

(51) Int. Cl. A61N 2/02; A61B 5/05

(52) U.S. Cl. CPC A61N 2/02; A61B 5/0507

(57) ABSTRACT

The present invention generally relates to a method and system of DNA resonance and its applications. In particular, the present invention provides a method and system of DNA resonance measurement which is done non-chemically. The invention is a device using an impedance spectroscopy and analyzing it to produce the diagnostic profile of the health and using initial treatments to see how the profile change and then adjusting the treatment to improve the profile in a specific direction, adjusting the parameters of the electric, electromagnetic and sound treatment for better outcomes. In this invention, the initial treatment is at 42.2 Gigahertz, which leads to Alu sequence decompaction and activation of the DNA leading to the improved biological outcomes, improved coherence and orderliness of the DNA and of the DNA fractal patterns. Further, the present invention is intended to various applications such as in therapy, communication to and from the body, communication to and from the brain, biotechnology and biological research using electromagnetic waves, patterned electric voltage, magnetic pulses, sound or other such means of stimulation applied individually and/or in combinations. Moreover, providing an algorithm of the conversion of DNA sequence into and from the wave patterns.

### FIELD OF INVENTION

This invention relates generally to the field of electromagnetic interactions with biological materials at the cellular, molecular, and sub-molecular levels, namely a method and system of DNA resonance and its applications. More specifically, the present invention disclosesa method and systemof DNA resonance measurement which is done non-chemically, usingDNA molecules and associated biological structures and processes pertaining to cellular generation, regeneration, and mitigation of degenerative conditions.The invention is a device using an impedance spectroscopy and analyzing it to produce the diagnostic profile of an individual's health and using initial treatments to see how the profile change and then adjusting the treatment to improve the profile in a specific direction, adjusting the parameters of the electric, electromagnetic and sound treatment for better outcomes.

### BACKGROUND OF THE INVENTION

Traditionally, life sciences, including biological, microbiological and genomic sciences, have primarily focused on electrochemical and physical properties and interactions at the cellular and molecular levels in biological materials in order to advance the study of disease detection, prevention, mitigation, and correction. While advances in such areas have had profound and undeniable beneficial impacts for humanity and non-human life for centuries, exploration of the electro-magnetic realm and its functional involvement in biological systems and processes remains a relatively new and uncharted domain.

DNA resonance treatments can be applied to therapy, performance improvement, performance improvement in fitness, research instrumentation, biotechnological production, food industry, agriculture, for brain computer interface (bidirectional exchange of information), use for synthetic telepathy, use for education, for the manipulation of the memory, for therapy of psychiatric disorders, for manipulation of the mind, for medical and non-medical adjustment of mood, emotions, sleep, brain activity, meditation, mental performance, studying performance, work performance, performance in unfavorable conditions, sports, fight, for performance under stress and for stress reduction.

inventors' studies and findings in the area of electromagnetic properties and processes of biological materials, and the mechanisms associated with DNA resonance in regards to cellular development in tissue generation and regeneration, represent significant discoveries which Inventors believe will prove foundational to the pursuit of novel methods in detecting and treating various conditions for human and non-human living things. Inventors believe such methods will have a profound impact on overall health, performance, quality, and longevity of life while helping to mitigate and even reverse degenerative conditions and disease states.

### DISCLOSURE OF PRIOR ART

A patent search conducted showed several patented inventions which are related to the art.

The concept of a DNA resonance and the application of this technology is already known. There are available patents which described the same. For instance, a patented invention described a method for readily and efficiently determining resonant frequencies that can be used therapeutically or beneficially, for debilitation of specific types of genomic materials, including DNA and/or RNA, genes, and gene sections. The methods can be used in a variety of circumstances related to various human and animal diseases and conditions. Methods allow the determination of therapeutic resonant frequencies for use in media having various refraction properties (U.S. Pat. No. 7,280,874).

Meanwhile, another patent disclosed according to some embodiments, an electromagnetic resonance-based disease treatment system that comprises a processing unit configured to generate a resonant frequency signal and a radiating antenna configured to radiate an electromagnetic field based on the resonant frequency signal. The resonant frequency signal may carry at least one frequency at which reference materials related to a disease condition resonate (U.S. Pat. No. 9,610,458).

Moreover, another patent presented a binodal lemniscate crown that alters abnormal brain synchrony patterns observed in autistic individuals establishing a harmonized flow of electromagnetic energy in the neural pathways that provides relief for headaches, reduces or eliminates various symptoms of autism, enhances memory and ability to focus; the said crown consisting of three conjoined circles that are formed from a piece of copper wire by bending it (CA Patent No. 2682098A1).

The WO Patent No. 1999002217A1 also described an invention which relates to the medical field, and in particular to impulse magneto therapy of biological objects. The therapeutic effect is achieved by concurrently subjecting the organism or/and the treated organ of the patient to the acoustic effect. As the acoustic effect there is applied, harmonious sounding producing stimulating effect. Furthermore, another patent also presented an apparatus and a method for therapeutically treating human body tissue with electromagnetic radiation (U.S. Pat. No. 5,723,001). Moreover, the U.S. Pat. No. 6,770,042 B2 also disclosed an invention for treating diseases with resonance generating electromagnetic fields. Multiple frequency bands with nonlinear frequency variation are combined to enhance the effectiveness of the therapy delivered by an infrasonic transducer or other delivery mechanism.

Furthermore, another patent disclosed a therapeutic method and apparatus to induce the healing of tissue cells by an electrical stimulus to a tissue area with the problematic condition by the application of low density charges of alternate polarities. The apparatus performs the therapeutic function by alternately charging and discharging the cells in 15-minute intervals over periods of time from 1 to 12 hours (U.S. Pat. No. 6,016,450).

Another patent also described a DNA Telomere rejuvenation. A copper screen placed between two one inch foam pads and covered with crushed sapphire crystal is connected to an activated Tesla coil. This leads to increased telomere length in normal white blood cells and can be used to increase the reproductive capacity of cells and to delay the onset of cellular senescence (US Patent No. 2011/0077727).

The WO Patent No. 2016/000075A1 also described a device, method and system for the microwave resonance therapy of chromosomes, telomeres and DNA and extending the length of telomeres, by generating a wide and uniformly distributed spectrum of electromagnetic frequencies and ultrasound vibrations that induce electromagnetic and ultrasonic resonance in chromosomes and telomeres and electromagnetic resonance in DNA. Moreover, another patent presented an invention which is a method and apparatus for applying low energy, non-ionizing, non-thermal electromagnetic radiation or electric current to the body of the subject for therapeutic and health promoting purposes (WO Patent No. 00/15295).

Another patent also described a resonance system for use on a subject having a biological pathology. The system includes a wave generator operably disposed adjacent a subject for generating a wave (which can be an electric wave) at a predetermined frequency, a resonant frequency sensor and generator operably disposed adjacent the subject for sensing a resonant frequency of a predetermined area of the subject in response to the predetermined frequency and generating a resonant frequency signal in response to said sensed resonant frequency, and a device (which is preferably computer based) operably associated with the sensor for receiving the resonant frequency signal and manipulating the resonant frequency signal in a manner to be displayed (US Patent No. 20040068168A1).

Another method was also disclosed intended for measuring in real-time the frequency and the power of an electromagnetic (EM) field emitted by a human body for different body conditions, the resonance phenomenon between the EM fields emitted by various products or electronic devices and the EM field emitted by a human body, and the signals emitted by active materials, waters, topical products, etc. A first real time spectrum analyzer is connected to a broad frequency range antenna placed in contact with the skin, electromagnetic fields emitted by a particular condition of the subject human body are measured to reveal peaks of power, and a second narrow frequency antenna is used to measure the peak more accurately (WO2010039465A2).

While there are available patents which described DNA resonance applications, most of these inventions are focused in treating diseases with resonance generating electrical or electromagnetic fields. There has been no method and system that provides a device which is non-destructively affecting DNA and/or receiving information from DNA in live cells via non-chemical means. Moreover, an algorithm for the conversion of DNA sequence into the wave patterns has not also been described in available patents. Apparently, this system and method applied for patent is new to the art and is found innovative which proves useful in the field of DNA resonance and applications. The present invention is directed towards this new and innovative idea.

Referring to “non-chemical cell-cell communication” effects, for instance, most basic experiments on biological fields involved two samples such as cell culture aliquots in sealed quartz cuvettes separated by optical filters. When one of the aliquotes is perturbed, the second one may catch the signal that is transferred non-chemically and is blocked by light impermeable filters. This refers to “non-chemical cell-cell communication” effects.

Meanwhile, although the idea that morphogenic field is holographic and is produced by the genomic sequence has been around since 1973 (Miller et al., 2012), there has been no attempts to decipher the algorithm of the conversion of DNA sequence into the wave patterns. Here, Inventors name this algorithm as “DNA resonance code” and define it as an algorithm which describes the conversion of genomic DNA sequence into the structure of the morphogenic field and ultimately to the shape of the body. Inventors believe that the DNA resonance code is not only the algorithm that reads the DNA structure and converts it into the wave structure, but that the process is two-directional—the genome receives the information via wave resonance and converts it into the structural information of the DNA by epigenetically condensing and de-condensing chromatin, modifying chromatin's chemistry and thus recording the received wave signals into the chemical structure.

Hence, it is an object of the present invention to provide a unique method and system of DNA resonance and its applications. In particular, a device that is non-destructively affecting DNA and/or receiving information from DNA in live cells via non-chemical means. Applications of the device to therapy, communication to and from the body, communication to and from the brain, and biotechnology and biological research using electromagnetic waves, electric voltage varied in time, magnetic pulses, sound or other such means of stimulation applied individually and/or in combinations. Moreover, by providing an algorithm of the conversion of DNA sequence into the wave patterns.

Although theories regarding the role of sequence-specific DNA resonance in biology have abounded for over 40 years, the published evidence for it is lacking. Here, the authors reasoned that for sustained resonance signaling, the number of oscillating DNA sequences per genome should be exceptionally high and that, therefore, genomic repeats of various sizes are good candidates for serving as resonators. Moreover, it was suggested that for the two DNA sequences to resonate, they do not necessarily have to be identical. Therefore, the existence of sequences differing in the primary sequence but having similar resonating sub-structures was proposed. It was hypothesized that such sequences, named HIDERs, would be enriched in the genomes of multicellular species. Specifically, it was hypothesized that delocalized electron clouds of purine-pyrimidine sequences could serve as the basis of HIDERs. The consequent genomic analysis confirmed the enrichment of purine-pyrimidine HIDERs in a few selected genomes of mammals, an insect, and a plant, compared to randomized sequence controls. Similarly, it was suggested that hypothetical delocalized proton clouds of the hydrogen bonds of multiple stacked bases could serve as sequence-dependent hydrogen-bond-based HIDERs. Similarly, the enrichment of such HIDERs was observed. It is suggested that these enrichments are the first evidence in support of sequence-specific resonance signaling in the genome.

Ninety-seven years ago, Alexander Gurwitsch proposed the existence of a morph ogenetic field that is created by the body and is responsible for developing and maintaining the shape of the body (Gurwitsch, 1922). He and others demonstrated that biological organisms influence the development of each other at short distances and that some of this influence is blocked by optical filters, suggesting that the morphogenic field is of an electromagnetic nature (Gurwitsch, 1988; Volodyaev and Beloussov, 2015). In 1968, Frohlich predicted that in the presence of constant energy flux, cell and organelle membranes produce coherent waves in the millimeter-wave region, thus creating a coherent state and enabling electric wave signaling in living organisms (Frohlich, 1988). In 1973, Miller and Web further proposed that it is DNA that is producing the morphogenic field and that the genomic code is directly sending and receiving the information from the morphogenic field (Miller and Webb, 1973). The experiments verifying the existence of biological fields involve two samples such as cell culture aliquots in sealed quartz cuvettes separated by optical filters. When one of the aliquots is perturbed, the second one may catch a signal that is transferred non-chemically and is blocked by light-impermeable filters. Such effects are often referred to as “non-chemical cell-cell communication” and are reviewed in refs (Cifra et al., 2011; Scholkmann et al., 2013; Trushin, 2004; Xu et al., 2017). Burlakov experimentally demonstrated that the optical distortion by quartz retroreflectors of the field produced by fish embryos causes developmental abnormalities, thus confirming that the field is morphogenic and electromagnetic (Burkov et al., 2008; Burlakov et al., 2012).

Although the existence of the field and its morphogenic and electromagnetic nature have been demonstrated, the involvement of DNA in its generation, proposed in 1973 by Muller and Webb, remains unproven. Many models for oscillations in DNA have been proposed that involve the movement of groups of atoms in DNA (referred to here as mechanical oscillations) (Scott, 1985; Volkov and Kosevich, 1987). The spectroscopic detection of coherent mechanical oscillations in DNA was reported to be in the THz range (Sajadi et al., 2011). Inventors proposed that in addition to mechanical oscillations in DNA, the base stack displays oscillations of delocalized electron clouds (Polesskaya et al., 2018), as well as of delocalized proton clouds of the hydrogen bonds (Savelyev et al., 2019). Moreover, inventors suggested that these oscillations occur in a DNA sequence-dependent manner and provide the primary medium for the formation of the morphogenic field. Inventors also suggested that since electron and proton clouds have low mass and are located inside the base stack, they do not cause significant movement of the heavier DNA atoms and the surrounding water, thereby avoiding the thermal dissipation of energy. Inventors suggested that, therefore, the electron and proton cloud oscillations in the base stack are a more likely medium for the morphogenic field than the heavier atoms of DNA (Polesskaya et al., 2018; Savelyev et al., 2019).

The inventors further proposed that electroacoustic resonances between similar DNA sequences form the basis of signaling within the genome and coordinate the function of the cell. Inventors also suggested possible mechanisms by which these oscillations are channeled by the microtubules from one nucleus to another, forming an oscillation network of the body. This way, Inventors transformed an idea of a diffuse morphogenic field into a model of the morphogenic field traveling between the nuclei via tunnels. This also explains how nature may avoid the dissipation of the electroacoustic signals in tissues (Savelyev et al., 2019). Inventors further implicated genomic repeats as primary candidate sequences to serve as resonators. Inventors suggested that since the 300 base pair-long Alu repeat occurs 1.1 million times in each of our cells, it is the ideal candidate for serving as a resonator by the mere number of copies improving the sustainability of oscillations and reducing the dissipation of the signal. Inventors also suggested that the primary function of genomic repeats such as telomeric, centromeric, simple repeats, and transposable elements is to support the resonance signaling in the genomes of complex organisms. Furthermore, Inventors proposed (Savelyev et al., 2019) that this resonance signaling system is deliberately supported by the cells via the flux of ATP and other biochemical energy, in accordance with the Frohlich models (Fröhlich, 1968). Inventors suggested that similar DNA sequences resonate with each other, forming a resonating network within the nucleus, between the nuclei and across the organism. During this process, some of the repetitive sequences may be energized by chemical processes, and their oscillations may be transmitted along the base stack, causing oscillations in similar sequences. This way, conformational changes in the chromatin in one location can lead to conformational changes in the chromatin of similar DNA sequences, allowing for resonance signaling within the nucleus, and across the organism. Inventors suggested that this process is deliberate, developed by evolution for higher organisms, and that the cell spends ATP and other types of chemical energy on supporting this resonance genomic signaling. This way, the chromatin immediately and mechanistically mediates the interaction between the electromagnetic resonance signaling and molecular signaling in a DNA sequence-specific manner. During this signaling process, the resonance properties of the DNA sequences provide specificity, while the ATP energy allows for the amplification of electromagnetic resonance signals and their conversion to molecular signals. For example, oscillations in some Alu sequences may be induced by ATP-dependent chromatin remodeling factors, and these oscillations may be transmitted via the base stack to the second group of Alu elements. Via electromagnetic resonance, the Alu elements of the second group begin resonance oscillation, which is amplified by the ATP-dependent chromatin remodeling factors bound to them, causing chromatin opening and transcription. This proposed mechanism would explain why Alu elements are enriched in the gene promoters (Savelyev et al., 2019).

Although there are experimental demonstrations of morphogenic field effects, to Inventors' knowledge, the involvement of DNA in its formation is yet to be proven. The prediction of oscillation frequencies in DNA is not trivial since it is likely that DNA could support several oscillation modes, including those of mechanical, electron and proton clouds. Since DNA is wrapped around nucleosomes, the chromatin state should also be considered. Inventors suggest that sequence-specific oscillations in DNA can spread over a wide range of frequencies. (FIG. 1)

Some insight might be obtained from electromagnetic frequencies used in physical therapy. Especially informative would be those frequencies, which produce effects at extremely low power, suggesting that they tap onto electromagnetic resonance signaling. Such frequencies are shown in Fig. [Spectrum]. Specifically, the following therapeutic ranges of electromagnetic frequencies exhibit significant effects at low power, and thus are likely to be tapping existing signaling pathways: pulsed electromagnetic field therapy (Binder et al., 1984), ultra high-frequency therapy (Lushnikov et al., 2004), millimeter wave therapy (Usichenko et al., 2003), low-level light therapy (Bjordal et al., 2003), and UVB (Lowe et al., 1991). Inventors suggest that these frequencies are good candidates for resonance oscillations in DNA. Since the frequency depends on the mass of the oscillator, shorter DNA repeats should oscillate at higher frequencies than the longer ones. Based primarily on these assumptions, Inventors propose the following approximate prediction of resonance frequencies of the genomic repeats, Table [Wavelengths]. Note that the natural wavelength of the oscillator can be much larger than its size. Recently, radio communication saw the development of nanomechanical magnetoelectric (ME) antennas, which resonate at wavelengths 1000 times larger than their size (Nan et al., 2017; Shi et al., 2016). An additional conversion factor is that electromagnetic oscillations are coupled with the acoustic oscillations in biological tissues. Therefore, an electromagnetic wavelength from a therapeutics device may be converted to an acoustic wave in the tissue, thus shortening the wavelength approximately 200,000 times. Although the predictions in Table [Wavelengths] are preliminary and requires experimental testing, they aim to illustrate the possible mechanistic connection between electromagnetic therapies and the proposed resonance genomic signaling. (FIG. 2)

Since so far, to Inventors' knowledge, there has been no published evidence for resonance genomic signaling, Inventors attempted to prove it computationally.

### OBJECTIVES OF THE INVENTION

The general purpose of the present invention is to provide a unique method and system of DNA resonance and its applications.

One significant objective of the present invention is to provide a device that is non-destructively affecting DNA and/or receiving information from DNA in live cells via non-chemical means.

Another objective of the present invention is to provide applications of the device to therapy, communication to and from the body, communication to and from the brain, and biotechnology and biological research using electromagnetic waves, electric voltage varied in time, magnetic pulses, sound or other such means of stimulation applied individually and/or in combinations.

Another objective of the present invention is to providean algorithm of the conversion of DNA sequence into the wave patterns.

These and other objects and features of the present invention will be apparent from the following detailed description, taken with reference to the figures of the accompanying drawing.

### SUMMARY OF THE INVENTION

The present invention provides a method and a device for diagnostics and therapy based on DNA resonance, a method for locating resonating DNA sequences in the genome, and a method for identifying the properties, structures, mechanisms, and frequencies of electromagnetic resonance in DNA molecules.

The present invention therefore generally relates to a DNA resonance and its applications. In particular, a device that is non-destructively affecting DNA and/or receiving information from DNA in live cells via non-chemical means. Applications of the device presented in this invention to therapy, communication to and from the body, communication to and from the brain, and biotechnology and biological research using electromagnetic waves, electric voltage varied in time, magnetic pulses, sound or other such means of stimulation applied individually and/or in combinations. In DNA resonance measurement, the information is obtained electromagnetically, electrically, acoustically, opto-acoustically or electro-acoustically. DNA resonance measurements can be utilized for diagnostics, non-diagnostics information gathering, identification of humans and livestock, or pets identifying those of the status and identity to individual readout of internal processes and by the technological process. This invention can be applied in the treatment of various infectious diseases, infectious and non-infectious disorders, cancers, brain-related and psychological conditions, including dementia, Alzheimers. Autism, Schizoprenia, depression, and various forms of addiction as well as mechanical injury and physical trauma, and regenerative therapies including organ regeneration or repair, and/or brain computer interface (bidirectional exchange of information), and for synthetic telepathy. These are indicative, and not intended to be exhaustive with regard to fields of beneficial application.

### BRIEF DESCRIPTION OF THE DRAWINGS

A clear understanding of the key features of the invention summarized above may be had by reference to the appended drawings, which illustrate the method and system of the invention, although it will be understood that such figures depict preferred embodiments of the invention and, therefore, are not to be considered as limiting its scope with regard to other embodiments which the invention is capable of contemplating. Accordingly:

FIG. 1 illustrates [Spectrum] Frequency ranges used for therapy. (LLLT—low-level light therapy, PEMF—pulsed electromagnetic field).

FIG. 2 show [Table] the very approximate prediction of resonance wavelengths of genomic repeats.

FIG. 3 shows the recoding schemes used.

FIG. 4 shows the effect of sequence randomization on the HIDER counts.

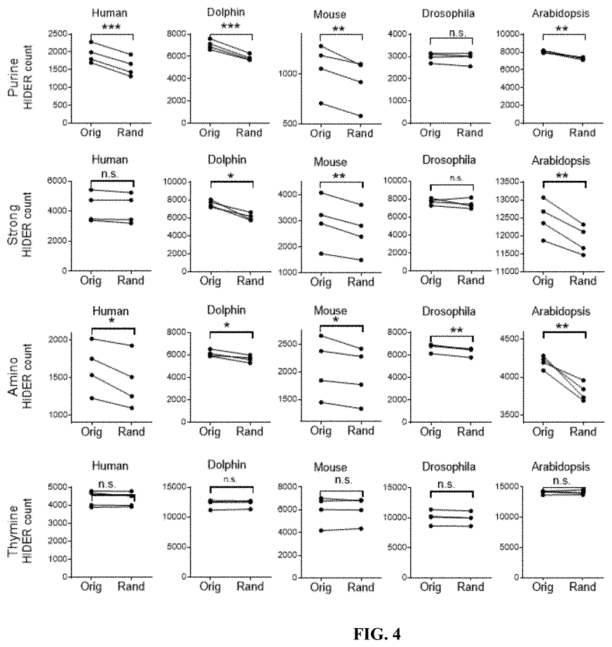

FIG. 5 shows the enrichment of HIDER counts in the original sequences over randomized sequences. (*—P<0.05, **—P<0.01, ***—P<0.001, n.s.—nonsignificant)

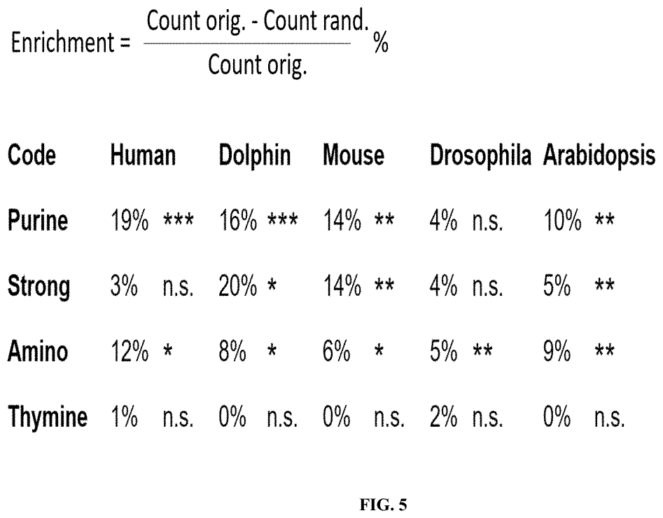

FIG. 6 shows the length dependence of Purine HIDER enrichment in arabidopsis.

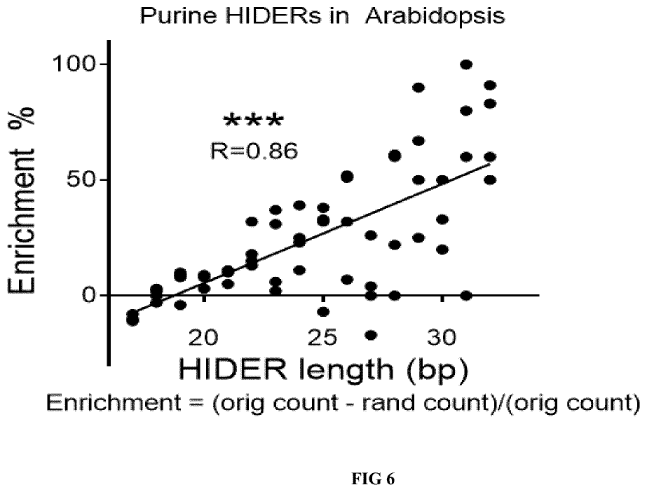

### DETAILED DESCRIPTION OF THE INVENTION

Unless otherwise defined, all terms (including technical terms) used herein have the same meaning as commonly understood by one having an ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques are disclosed. Each of these has individual benefits and each can also be used in conjunction with one or more, or in some cases all, of the other disclosed techniques. Accordingly, for the sake of clarity, this description will refrain from repeating possible combination in an unnecessary fashion. Nevertheless, the specifications and claim/s should be read with the understanding that such combinations are entirely within the scope of the invention and the claim/s.

The present disclosure is to be considered as an exemplification of the invention and is not intended to limit the invention.

The invention is a method and system of DNA resonance and its applications.

In particular, this invention is a device which is non-destructively affecting DNA and/or receiving information from DNA in live cells via non-chemical means. It is also a device where one or more of the following is used: electromagnetic waves, electric voltage varied in time, magnetic pulses, sound or other such means of stimulation applied individually and/or in combinations.

According to a preferred embodiment of the present invention, the device uses impedance spectroscopy and analyzing it to produce the diagnostic profile of an individual's health and using initial treatments to see how the profile change and then adjusting the treatment parameters field for improved outcome.

According to a preferred embodiment of the present invention, the invention is a device used for initial treatment at 42.2 Gigahertz, which leads to Alu sequence decompaction and activation of the DNA improved biological outcomes, improved the coherence and orderliness of the DNA and of the DNA fractal patterns. Moreover, the inventors have suggested that the main function of Alu is vibrational and proposed that Alu is responsible for the creation of the uniquely human (primate) morphogenic field and that is the key resonating component of our mind and consciousness. Also, the inventors believe that Alus are creating the main part of the field in the nucleus and while interacting with the field they make the major contribution in the control of which genes are transcribed.

According to a preferred embodiment of the present invention, the DNA sequence-specific activation and/or repression of certain DNA sequences are achieved.

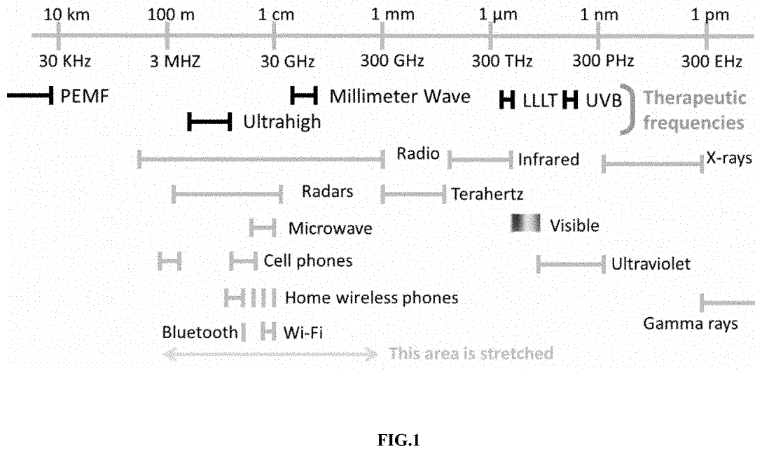

According to a preferred embodiment of the present invention, the device is used for application to therapy, communication to and from the body, communication to and from the brain, biotechnology and biological research. On communication to and from the body, referring to FIG. 1, it was proposed that the microtubules (101) and DNA communicate resonantly through the nuclear membrane (102), and that the microtubules of neighboring cells communicate (104) resonantly through the contact points of the cell membranes (103), thereby integrating all the body nuclei by the waveguides. From this, it follows that resonant vibrations of DNA propagate not by chance, but are guided by waveguides of microtubules. It was also proposed that the nervous system and connective tissue are primarily responsible for uniting the organism into one resonating system.

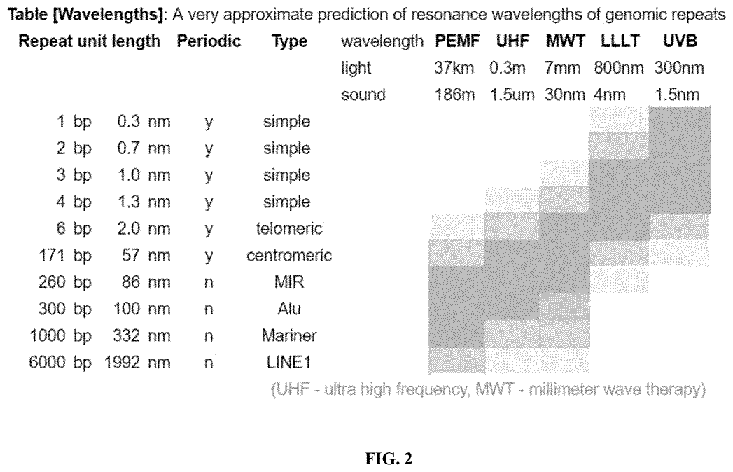

According to a preferred embodiment of the present invention, referring to FIG. 2, it was also proposed that in neurons (203, 204), the propagation of action potential (201) across the axons is the process of reading and writing of information into and from microtubules (205) and that this propagation of action potential is electromagnetically connected (202) via the microtubules with the DNA in the nucleus thus allowing the participation of DNA in the work of mind and memory.

According to a preferred embodiment of the present invention, another feature of this invention is the method and system where the sequential signals is patterned in time.

According to a preferred embodiment of the present invention, another embodiment of this invention is the method and system where spatially patterned signals are produced. Spatially patterned signals, the shape of the wave, which is its circular polarization, chirality and its 3-dimensional structure.

Furthermore, another embodiment of this invention is the method and system of obtaining the patterns via bio-impedance spectroscopy, analyzing them, interpreting and returning to the body as DNA resonance treatment, in accordance with obtained information.

Meanwhile, another embodiment of this invention is a method and system of obtaining an electromagnetic signature from DNA samples, or living tissues or living bodies and recording it, amplifying and returning back to activate or repress specific DNA sequence groups for therapy, biotechnology and other applications.

Moreover, another embodiment of this invention is the process of starting the treatment, then observing the changes via DNA resonance diagnostics and adjusting the treatment accordingly to improve the diagnostic results; which can be done is real time or in alternating steps during treatment and diagnostic.

Finally, another unique embodiment of this invention is the method where biofeedback is based on electric measurements, electromagnetic measurements, sound measurements, temperature measurements, optical measurements, optoacoustic or electroacoustic measurements, measurements of temperature, detailed pulse characteristics, bio-impedance spectroscopy, a response of the above measurements to active perturbation, and/or measurements performed at specific points including acupuncture points and Zakharyin-Ged Zones.

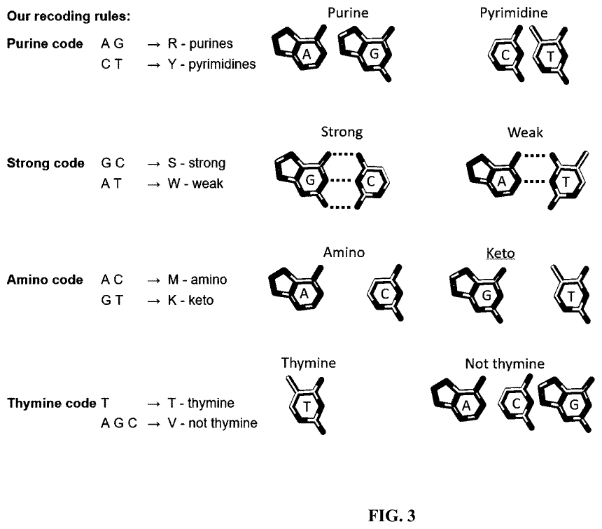

According to a preferred embodiment of the present invention, since Inventors believe that the majority of repetitive sequences in the genome are involved in meaningful, resonance signaling, Inventors hypothesized that some of the unique (non-repetitive) sequences in the genome might have evolved to resonate with the genomic repeats. Accordingly, Inventors hypothesized that it is not necessary for the unique sequence to be identical to the repeat, that for resonance, it might need to be only partially similar to the sequence of the repeat: for example, it is possible that some oscillations involve primarily the electron clouds of the aromatic rings (Savelyev et al., 2019). Therefore, only the purine-pyrimidine structure of the resonating sequences should be similar, while their primary sequences can be different. This simplification of the sequence from the primary sequence to the purine-pyrimidine sequence, is hereafter referred to as the “Purine code.” Similarly, for the oscillations primarily involving the hypothetical clouds of the delocalized protons of the hydrogen bonds in base pairs, only the patterns of these bonds should be similar, while the primary sequence can be different. This simplification of the sequence from primary to strong/weak (three bonds/two bonds per base pair), is hereafter referred to as the “Strong code.” The recoding rules used here are listed in FIG. 3.

Similarly, Amino and Thymine codes were used in the analysis. Therefore, Inventors attempted to search for sequences that are unique (non-repetitive), but become similar to genomic repeats or each other after recoding (simplification). Inventors will refer to them as HIDERs (Homologous If Decoded Elements, Repetitive). In accordance with the four recoding schemes, Table [Codes], four types of HIDERs were analyzed: Purine, Strong, Amine, and Thymine. On the primary sequence level, HIDERs are unique (non-repetitive) sequences, which are identical to each other after recoding. On the physical level, Inventors expect these to be engaged in resonance signaling, and therefore, enriched in the genomes of complex organisms. One of the advantages of such a computational genomics approach is that it is agnostic to the exact physical mechanism of the resonance, allowing verification of its existence prior to the discovery of the mechanism. Once HIDERs are found, their chemical structure may provide an insight into the modes of their resonance.

Methods

The repeats were masked using RepeatMasker (http://repeatmasker.org/) followed by a heuristic removal of repeats using Ugene 1.32.0 (http://ugene.net/). Recoding was done as described in Fig. [Codes]. HIDERs were detected by searching for similar pairs of fragments in the recoded sequences using Ugene, analyzed in Google Sheets, and plotted with GraphPad Prism. Randomized sequences were used as controls, see Supplement. To retain the distribution of nucleotide densities along the sequence, randomization was done only on the unmasked parts of the sequence within each 20-nucleotide bin. The significance of enrichment was determined using the t-test.

Results

HIDERs are enriched in genomes compared to randomized controls.

The inventors selected five species for analysis. In addition to humans, Inventors chose mice, drosophila, and arabidopsis as typical model species and the dolphin as a highly developed aquatic mammal.

In each genome, four 90 kb pieces were selected at random, and the repeats were masked. Randomized reference sequences (RAND) were created from the original sequences (ORIG). ORIG and RAND sequences were recoded, as presented in Table [Codes]. In each sequence, pairs of identical strings (HIDERs) longer than 19 bases were identified.

Among the five tested species, the highest enrichment of HIDERs was found in mammals and the lowest in drosophila. Among the recoding schemes, the highest enrichment was found in the Purine code and the lowest in the Thymine code. The highest statistical significance was observed for the enrichment of Purine HIDERs in humans and dolphins.

Length Dependence

In arabidopsis, the Purine HIDERs demonstrated a positive correlation of the HIDERs' enrichment with their length the enrichment was higher for longer HIDERs.

This suggests that longer HIDERs might be functional and, thus, preferentially selected during the process of evolution. Such correlation was less pronounced in the other species studied.

Discussion

As detailed in the Introduction, Inventors' initial motivation was to find sequence-dependent DNA resonators. Inventors realized that for resonance to be sustained, the number of DNA resonators needs to be very high in each cell, in the order of millions of copies. Logically, so-called “junk DNA” made of repetitive elements of various sizes, is the primary candidate for harboring DNA resonators. Inventors suggested that the key resonator in the human genome is the Alu element, which is represented by 1.1 million copies per cell. Then, Inventors proposed that since DNA resonators ought to serve a function in coordinating the operation of the cell and the transfer of information between cells, resonator sequences should evolve to be enriched in the genome. Moreover, inventors hypothesized that even non-repetitive (unique) sequences might resonate with the repetitive sequences if they support similar modes of oscillation, that is, similar frequencies and patterns of electromagnetic oscillations. Then, Inventors looked specifically for chemical structures in the DNA, which might support sequence-specific oscillations, and suggested that purine-pyrimidine patterns might be characterized by unique vibrational patterns. Specifically, Inventors hypothesized that the pi-electrons of aromatic rings of multiple nucleobases might form a collective delocalized electron cloud, shape and oscillation pattern, of which would be defined primarily by the purine-pyrimidine sequence. Therefore, DNA sequences, having a different primary sequence but common purine-pyrimidine patterns, might resonate. Inventors called such sequences HIDERs, and suggested that they might be enriched in the genome. Here, Inventors tested this hypothesis and confirmed the enrichment of the HIDERs in the selected mammal species and arabidopsis but not in drosophila, Fig. [Enrichment].

Similarly, Inventors hypothesized that protons of hydrogen bonds of neighboring base pairs would form a delocalized proton cloud (a proton highway). This cloud would be prone to oscillations, and these oscillations would depend on the DNA sequence, specifically on the order in which base pairs with two hydrogen bonds (weak: A, T) and three hydrogen bonds (strong: C, G), respectively, occur in the DNA sequence. As above, Inventors tested whether Strong HIDERs would be enriched in the genome. Inventors observed a significant enrichment in the dolphins, the mice, and arabidopsis, but not in humans or drosophila, Fig. [Enrichment].

Note that both the Purine and Strong codes are simplifying the sequence from fInventors' symbols (A, C, G, T) to two symbols (purine/pyrimidine or strong/weak). Some information, including side radicals of the nucleobases, is lost, presumably allowing the HIDERs of different primary sequences to resonate with each other and likely with high-copy genomic repeats. Although Inventors believe that high-copy genomic repeats are the primary resonators in the cell, Inventors focused here on HIDERs since they allow the inventors to test the DNA resonance hypothesis via computational genomics.

To reiterate, to Inventors' knowledge, this is the first, although indirect, evidence of DNA resonance in biology. However, although the obtained evidence is encouraging, more research is needed to verify the existence and the mechanistic details of DNA resonance. Computational modeling of the proposed electron and proton clouds of DNA sequences with the use of methods of quantum chemistry and structural biology could verify and substantiate the existence of such sequence-dependent resonating structures. Spectroscopic measurements could substantiate the proposed resonances between various sequences, including the ones highlighted by Inventors' analyses.

The inventors are aware that in addition to the DNA resonance explanation, there are possible explanations for the observed enrichments that could involve traditional chemical causes. For example, it is possible that purines are more likely to mutate into each other than into pyrimidines, and vice versa. Therefore, repetitive sequences via the process of mutation might diverge in their primary sequence while retaining their purine-pyrimidine sequence, thus, effectively becoming HIDERs. Similarly, certain repeats might be the targets of transcription factors, which recognize their strong-weak pair sequence while ignoring actual bases. Consequently, certain repeats might diverge in evolution, producing Strong HIDERs. Currently available evolutionary base-substitution rates are not precise enough to enable the delineation of the chemical and resonance causes for the enrichment signal of HIDERs. Therefore, Inventors hope that Inventors' results encourage further research and the hypothesis of sequence-dependent DNA resonance signaling will be verified more conclusively.

One of the challenges in using computational genomics as a tool for testing hypotheses is the need for the Bonferroni correction in the case of multiple comparisons. To avoid multiple comparisons, Inventors randomly selected the DNA fragments only once, and did not optimize any analysis parameters. The selection and analysis of the data occurred only once. Moreover, to allow for testing the phenomena observed by others, Inventors selected only a few species and only a small part (approximately 3%) of each genome. This way, others could easily reproduce the observed enrichments on untouched data sets.

Supplement

Sequences used for the analysis.

Four original and four randomized sequences were investigated,

Random sequence selection method: To avoid the multiple comparison problem in the statistics, the selection of sequences was performed only once. Each sequence was 90 Kb long. The selection was achieved using a simple algorithm, and the coordinates were predetermined using a simple rule. The assemblies and the coordinates were as follows:

Human

hg38_dna range=chr1: 100000000-100090000

hg38_dna range=chr1: 100090001-100180000

hg38_dna range=chr1: 100180001-100270000

hg38_dna range=chr1: 100270001-100360000

Dolphin

turTru2_dna range=JH472452:10000-100000

turTru2_dna range=JH472452:181000-271000

turTru2_dna range=JH472452:309250-399250

turTru2_dna range=JH472452:480250-570250

Mouse

mm10_dna range=chr3:32500000-32590000

mm10_dna range=chr3:32590001-32680000

mm10_dna range=chr3:32680001-32770000

mm10_dna range=chr3:32770001-32860000

Drosophila

dm6_dna range=chr2L:200000-290000

dm6_dna range=chr2L:400000-490000

dm6_dna range=chr2L:800000-890000

dm6_dna range=chr2L:1200000-1290000

Arabidopsis

hub_329263_araTha1_dna range=chr3:400000-490000

hub_329263_araTha1_dna range=chr3:600000-690000

hub_329263_araTha1_dna range=chr3:1490000-1580000

hub_329263_araTha1_dna range=chr3:1800000-1890000

Repeat Masking

Repeat masking was conducted in two steps. The original sequence was uploaded into the online RepeatMasker service (http://repeatmasker.org/cgi-bin/WEBRepeatMasker), and the repeats were masked with Ns. Then the sequence was masked by the Find Repeats algorithm of the UGENE program (Unipro UGENE http://ugene.net/).

Search for HIDERs in the Recoded Sequence.

The masked sequence was randomized as described in the Methods section. The original and randomized sequences were transformed into degenerate codes, as shown in Fig. [Codes]. Pairs of identical HIDERs longer than 19 pairs were identified using the Find Repeats algorithm of the UGENE program. The accuracy of the search was tested in part by verifying that the primary source sequences for the pairs of HIDERs were different as intended.

The terminology used herein is for the purpose of describing particular embodiments only and is not intended to be limiting of the invention. As used herein, the term includes any and all combinations of one or more of the associated listed items. As used herein, the singular forms are intended to include the plural forms as well as the singular forms, unless the context clearly indicates otherwise.

While the preferred embodiments of the present invention has been shown and described in detail, it is to be understood that numerous modifications can be made to the preferred embodiment without departing from the spirit of the invention. Therefore, it should be clearly understood that the forms of the present invention described above and shown in the figures of the accompanying drawings are illustrative only and are not intended to limit the scope of the present invention.

While the preferred embodiments of the present invention has been shown and described in detail, it is to be understood that numerous modifications can be made to the preferred embodiment without departing from the spirit of the invention. Therefore, it should be clearly understood that the forms of the present invention described above and shown in the figures of the accompanying drawings are illustrative only and are not intended to limit the scope of the present invention.

We claim:

1. A method and system of DNA resonance and its applications, comprising of a device which is non-destructively affecting DNA and/or receiving information from DNA in live cells via non-chemical means characterized in that the device uses impedance spectroscopy and analyzing it to produce the diagnostic profile of an individual's health and using initial treatments to see how the profile change and then adjusting the treatment parameters field for improved outcome.

2. A method and system of DNA resonance and its applicationsas claimed in claim 1, wherein electromagnetic waves, electric voltage varied in time, magnetic pulses, sound or other such means of stimulation applied individually and/or in combinations is used in the device.

3. A method and system of DNA resonance and its applications as claimed in claim 1, whereinthe device used for initial treatment at 42.2 Gigahertz, which leads to Alusequence decompaction and activation of the DNA improved biological outcomes, improved the coherence and orderliness of the DNA and of the DNA fractal patterns.

4. A method and system of DNA resonance and its applicationsas claimed in claim 1, whereinthe device used for initial treatment at 42.2 Gigahertz, which leads to Aluand other repetitive elements sequence decompaction and activation of the DNA improved biological outcomes, improved the coherence and orderliness of the DNA and of the DNA fractal patterns.

5. A method and system of DNA resonance and its applications as claimed in claim 1, whereinthe main function of Alu and other repetitive elementsis vibrational and is responsible for the creation of the uniquely human (primate) morphogenic field and that is the key resonating component of our mind and consciousness.

6. A method and system of DNA resonance and its applications as claimed in claim 1, whereinAlus and other repetitive eiementsare creating the main part of the field in the nucleus and while interacting with the field they make the major contribution in the control of which genes are transcribed.

7. A method and system of DNA resonance and its applications as claimed in claim 1, whereinthe DNA sequence-specific activation and/or repression of certain DNA sequences are achieved.

8. A method and system of DNA resonance and its applications as claimed in claim 1, whereinthe device is used for application to therapy, communication to and from the body, communication to and from the brain, biotechnology and biological research.

9. A method and system of DNA resonance and its applications as claimed in claim 1, whereinon communication to and from the body, the microtubules (101) and DNA communicate resonantly through the nuclear membrane (102), and that the microtubules of neighboring cells communicate (104) resonantly through the contact points of the cell membranes (103), thereby integrating all the body nuclei by the waveguides.

10. A method and system of DNA resonance and its applications as claimed in claim 1, whereinresonant vibrations of DNA propagate not by chance, but are guided by waveguides of microtubules.

11. A method and system of DNA resonance and its applications as claimed in claim 1, whereinthe nervous system and connective tissue are primarily responsible for uniting the organism into one resonating system.

12. A method and system of DNA resonance and its applications as claimed in claim 1, whereinin neurons (203, 204), the propagation of action potential (201) across the axons is the process of reading and writing of information into and from microtubules (205) and that this propagation of action potential is electromagnetically connected (202) via the microtubules with the DNA in the nucleus thus allowing the participation of DNA in the work of mind and memory.

13. A method and system of DNA resonance and its applications as claimed in claim 1, whereinthe method and system where the sequential signals is patterned in time is claimed.

14. A method and system of DNA resonance and its applications as claimed in claim 1, whereinthe method and system where spatially patterned signals, the shape of the wave, which is its circular polarization, chirality and its 3-dimensional structure, is produced, is claimed.

15. A method and system of DNA resonance and its applications as claimed in claim 1, wherein the method and system of obtaining the patterns via bio-impedance spectroscopy, analyzing them, interpreting and returning to the body as DNA resonance treatment, in accordance with obtained information, is claimed.

16. A method and system of DNA resonance and its applications as claimed in claim 1, whereinelectromagnetic signature from DNA samples, or living tissues or living bodies is obtained and recording it, amplifying and returning back to activate or repress specific DNA sequence groups for therapy, biotechnology and other applications.

17. A method and system of DNA resonance and its applications as claimed in claim 1, whereinthe process of starting the treatment, then observing the changes via DNA resonance diagnostics and adjusting the treatment accordingly to improve the diagnostic results; which can be done is real time or in alternating steps during treatment and diagnostic, is claimed.

18. A method and system of DNA resonance and its applications as claimed in claim 1, whereinthe method where biofeedback is based on electric measurements, electromagnetic measurements, sound measurements, temperature measurements, optical measurements, optoacoustic or electroacoustic measurements, measurements of temperature, detailed pulse characteristics, bio-impedance spectroscopy, a response of the above measurements to active perturbation, and/or measurements performed at specific points including acupuncture points and Zakharyin-Ged Zones is claimed.

19. A method and system of DNA resonance and its applications as claimed in claim 1, whereinthe method locates purine-coded repetitive elements in DNA recoded into purine code (in which A and G are recoded to R; and C and T are recoded to Y) and target these-purine coded repetitive elements in physical therapy.

20. A method and system of DNA resonance and its applications as claimed in claim 1, whereinthe method locatesstrong-coded repetitive elements in DNA recoded into strong code (in which G and C are recoded to S; and A and T are recoded to W) and target these strong-coded repetitive elements in physical therapy.

21. A method and system of DNA resonance and its applications as claimed in claim 1, whereinthe method optimizes the field treatments by treating DNA samples or living biological objects with various treatments, measuring the effects on Alu and other repetitive elements and optimizing the treatment parameters to achieve optimal compaction or decompaction of Alu or other selected repetitive elements

22. A method and system of DNA resonance and its applications as claimed in claim 1, whereinthe method optimizes the field treatments by treating DNA samples or living biological objects with various treatments, measuring the effects on primary-coded, purine-coded and strong-coded repetitive elements and optimizing the treatment parameters to achieve optimal compaction or decompaction of selectedprimary-coded, purine-coded and strong-coded repetitive elements
